(19) United States
(12) Patent Application Publication
(10) Pub. No.: US 2021/0106757 A1
(43) Pub. Date: Apr. 15, 2021

(54) AUTOINJECTION DEVICE HAVING A MEMORY ELEMENT

(71) Applicant: Novo Nordisk A/S, Bagsvaerd (DK)

(72) Inventors: Hans Hemmingsen, Birkeroed (DK); Ebbe Kiilerich, Copenhagen NV (DK)

(21) Appl. No.: 17/044,153

(22) PCT Filed: Apr. 1, 2019
(86) PCT No.: PCT/EP2019/058212

§ 371 Date: Sep. 30, 2020

(30) Foreign Application Priority Data
Apr. 1, 2018 (EP) 18165358.5

(51) Int. Cl. A61M 5/20; A61M 5/32

(52) U.S. Cl. CPC A61M 5/2033; A61M 5/3202

(57) ABSTRACT

An autoinjection device (10) for expelling a dose of drug from a held drug container (100) is described. A plunger (500) is drivable by an energy source (550) for expelling a dose, the plunger (500) comprising a retaining geometry (515) in retaining engagement with a retaining element (410, 415), wherein the autoinjection device (10) defines a trigger element (700) serving as a memory element movable from a pre-firing position to a fired position, the memory element comprising an engagement surface (700c) configured for sliding engagement with an activation surface (415c) of the retaining element (410, 415), wherein at least one of the engagement surface (700c) and the activation surface (415c) includes a surface being inclined relative to said radial direction, and wherein the energy source (550) acts on the plunger (500) to force the retaining element (410, 415) radially to release the retaining engagement, the radial movement of the retaining element (410, 415) in turn forcing the memory element to move into the fired position.

### FIELD OF THE INVENTION

The present invention relates to injection devices for injecting a medicament. In particular, the present invention relates to an autoinjection device having a releasable plunger, and a memory element which controls a secondary function of the autoinjection device, wherein said secondary function is distinct from the function associated with the release of the plunger.

### BACKGROUND OF THE INVENTION

In relation to some diseases patients must inject a medicament on a regular basis such as once weekly, once daily or even a plurality of times each day. To help patients overcome fear of needles, some injection devices incorporate a needle shield or needle shroud for covering an injection needle either before or after a dose administration. Such needle shield or shroud typically hide the injection needle from the user's view, and in some forms, additionally provide a mechanical blocking preventing an individual from accidentally gaining access to the injection needle.

US 2017/0136192 A1 discloses an autoinjector which is configured for being triggered to inject a dose when a needle shield is moved from a distal extended position towards a proximal collapsed position. The needle shield cooperates with a trigger element, and both elements are moved in unison from the distal extended position into the proximal collapsed position. Subsequent to injection, a needle shield spring acts to push the trigger element and the needle shield towards a distal extended position. A needle shield lock incorporates a lock activator associated with an activation geometry arranged on a plunger.

WO 2001/32255 A1 discloses a needle assisted jet injector having a locking mechanism for reducing the likelihood of inadvertent contact with the needle and deterring intentional reuse of the needle. A locking member is associated with a needle guard in a first retracted position, is associated with a holder member in the extended and second retracted positions, and is in blocking relation with the needle guard in the second retracted position. The jet injector further includes a ram trigger mechanism which is also operated by the needle guard.

In connection with design and manufacture of autoinjection devices tolerance variations make multiple individual distinct functions that are operated by one and the same element difficult or even unreliable. For example, for an injector as disclosed in WO 2001/32255 A1 there is a risk that the needle guard will not become locked by the locking mechanism even though the ram has been triggered. Also, there is a risk that the ram will not become triggered even though the locking mechanism has been activated.

Having regard to the above-identified prior art devices, it is an object of the present invention to provide an autoinjection device that is improved having regard to performing multiple functions and wherein the device is less sensitive to tolerance variations.

Yet additional further objects of the invention are to provide measures for obtaining devices having a superior performance and, at the same time, enabling manufacture at a reduced cost.

### BRIEF DESCRIPTION OF THE INVENTION

In a first aspect the present invention relates to an autoinjection device for expelling a dose of drug from a held drug container, the injection device comprising:
a housing having a proximal end (P) and a distal end (D),
a drug container comprising a container barrel and a piston that is sealingly and slideably arranged inside the container barrel,
an injection needle connected to or connectable to a distal end of the drug container,
a plunger adapted for cooperation with the piston to drive the piston distally along a central axis, the plunger comprising a retaining geometry,
an energy source coupled to the plunger and providing a force on the plunger in a distal direction,
a plunger retaining arrangement comprising a retaining element that engages with the retaining geometry to retain the plunger in a pre-firing position, the retaining element being movable in a radial direction to release said engagement,
a user operable trigger element cooperating with the retaining element and shiftable from a pre-firing condition wherein the trigger element cooperates with the retaining element to maintain retaining engagement with the retaining geometry of the plunger, and into a firing condition wherein release of the retaining engagement is initialised,
   wherein the autoinjection device defines a memory element being movable from a pre-firing position to a fired position, the memory element comprising an engagement surface configured for sliding engagement with an activation surface of the retaining element, wherein at least one of the engagement surface and the activation surface includes a surface being inclined relative to said radial direction, and
   wherein, upon the trigger element being shifted from the pre-firing condition to the firing condition, the energy source acts on the plunger to force the retaining element radially to release the retaining engagement, the radial movement of the retaining element in turn forcing the memory element to move into the fired position by sliding engagement between the activation surface of the retaining element and the engagement surface of the memory element, said movement of the memory element being induced by said surface being inclined relative to said radial direction.

As the energy source acts to force the memory element to move into the fired position the autoinjection device becomes less dependent on tolerance variations so that the correct functions of a secondary function controlled by the position of the memory element becomes more reliable. In addition, the movement of the memory element into the fired position means that the movement of the memory element becomes less dependent on how the user operates the device.

In some embodiments, the memory element, when assuming the fired position, controls a secondary function of the autoinjection device, wherein said secondary function is distinct from the function associated with the release of the plunger, i.e. a function other than the function provided by the retaining element being moved in the radial direction to release the engagement with the retaining geometry of the plunger. A non-exhaustive list of secondary functions includes one or more of controlling initialisation or generation of a feedback signal represented by or triggered by the memory element assuming the fired position. The secondary function is conditional to the release of the plunger, and may be configured to provide a feedback signal such as a visible, audible or tactile signal, or configured to provide an electronic signal to be recorded or stored in electronic circuitry, wherein the electronic signal is responsive to the release of the plunger. Still other secondary functions may include a latch function, such as for locking a needle shroud in a particular position, such as a needle covering position, i.e. conditional to the release of the plunger.

In some embodiments the memory element is axially movable, and wherein said at least one surface is inclined relative to said radial direction and so oriented as to induce axial movement of the memory element from the firing position to the fired position upon radial movement of the retaining element.

In other embodiments the memory element is rotationally movable, and wherein said at least one surface is inclined relative to said radial direction and so oriented as to induce rotation of the memory element from the firing position to the fired position upon radial movement of the retaining element.

In some embodiments the retaining element includes a retaining surface that engages a cooperating surface of the retaining geometry to retain the plunger in the pre-firing position, and wherein one or both of the retaining surface and the cooperating surface include(s) a surface being inclined relative to said radial direction so that distal movement of the plunger, upon initial release of the retaining engagement, induces radial movement of the retaining element to disengage the retaining surface from the cooperating surface of the retaining geometry.

In some embodiments the trigger element assumes a pre-firing position when the trigger element assumes the pre-firing condition, and assumes a firing position when the trigger element assumes the firing condition, and wherein the trigger element cooperates with the retaining element to initiate release of the retaining engagement when the trigger element assumes the firing position.

In some embodiments the trigger element defines said memory element, and wherein the trigger element is movable from the pre-firing position to the firing position, and further to the fired position. In such embodiment, the force of the energy source acts to move the trigger element from the firing position to the fired position by said sliding engagement between the activation surface of the retaining element and the engagement surface of the memory element.

In some embodiments, the trigger element is movable axially from the pre-firing position to the fired position, such as movable proximally from the pre-firing position to the fired position. In some embodiments, the firing position is positioned at an intermediary position between the pre-firing position and the fired position.

In other embodiments, the trigger element is rotationally movable from the pre-firing position to the firing position. In some embodiments wherein the trigger element defines said memory element, the trigger element is rotationally movable from the pre-firing position to the firing position, and further rotationally movable into the fired position. In these embodiments, one or both of the engagement surface and the activation surface include(s) a surface being inclined relative to said radial direction and being so oriented that radial movement of the retaining element induces rotation of the trigger element into the fired position. The sequence of the rotation is initiated by the user which initially drives the trigger element to rotate from the pre-firing position to the firing position. Thereafter, the trigger element is in the first place urged to move by distal movement of the plunger, as forced by the energy source, which in turn induces radial movement of the retaining element, and which in turn induces rotation of the trigger element from the firing position to the fired position.

Some embodiments of the autoinjection device comprises a needle shroud being axially movable relative to the housing, and a needle shroud spring which is arranged biasing the needle shroud in the distal direction, wherein the needle shroud is movable from a first distal extended position into a proximal collapsed position when a proximally directed force is applied to the needle shroud, and from the proximal collapsed position into a distal extended locked position, and wherein the trigger element couples to the needle shroud so that the trigger element moves from the pre-firing position to the fired position in response to the needle shroud being moved from the first distal extended position into the proximal collapsed position.

In some embodiments, the first distal extended position is the same as the distal extended locked position. In other embodiments, the first distal extended position may be located distally or proximally relative to the distal extended locked position. The distal extended locked position defines a state wherein the needle shroud protects the needle from being touched by the user. The proximal collapsed position defines a state wherein the needle extends distally beyond the needle shroud, or where the needle is positionable to extend distally beyond the needle shroud, to allow for insertion of the needle into an injection site.

The needle shroud may be configured so that when it moves from the first distal extended position towards the proximal collapsed position the needle shroud causes the trigger element to move from the pre-firing position into the fired position, the needle shroud slaving the trigger element into the firing position, and optionally into the fired position. In some embodiments, the needle shroud engages the trigger element to slave the trigger element as the needle shroud moves from the first distal extended position towards the proximal collapsed position.

In some embodiments a latch is associated with the trigger element, the latch engaging when the trigger element assumes the fired position to arrest the trigger element in the fired position. The latch may be associated with the housing, and may be provided as a permanent latch.

The latch may in some embodiments be provided by cooperating latch geometries of the trigger element and the housing, such as a component fixedly associated with the housing, to prevent the trigger element from being moved distally away from the fired position. In other embodiments the latch is configured to rely on a frictional coupling, such as a frictional engagement, between the trigger element and the housing to prevent the trigger element from being moved distally away from the fired position. In some embodiments the latch is configured to provide a permanent axial locking of the trigger element relative to the housing when the trigger element assumes the fired position.

In some embodiments, when the needle shroud moves from the proximal collapsed position into the distal extended locked position, the needle shroud moves relative to the arrested trigger element, and wherein the needle shroud cooperates with the trigger element to lock the needle shroud as the needle shroud is moved distally into the distal extended locked position.

In some embodiments at least one of the needle shroud and the trigger element comprises a lock element which is resiliently urged towards the other of the needle shroud and the trigger element to move along relative to a surface of said other of the needle shroud and the trigger element when the needle shroud moves relative to the arrested trigger element until the lock element reaches a locking geometry formed in or on said other of the needle shroud and the trigger element upon the needle shroud being moved distally into the distal extended locked position so as to lock the needle shroud in the distal extended locked position.

In some embodiments the trigger element includes a distally directed lock surface configured to engage a proximally directed locking geometry of the needle shroud to prevent the needle shroud to be moved proximally when the needle shroud assumes the distal extended locked position.

In some embodiments the distally directed lock surface is formed on a resiliently movable lock element being movable from a non-locking position into a locking position, and wherein a biasing means urge the resiliently movable lock element towards moving to the locking position, and wherein the resiliently movable lock element is moved from the non-locking position into the locking position upon the needle shroud being moved from the proximal collapsed position into the distal extended locked position.

In some embodiments the resiliently movable lock element is configured to slide along a sliding surface of the needle shroud as the needle shroud is moved from the proximal collapsed position into the distal extended locked position for the distally directed lock surface of the trigger element to axially align with the proximally directed locking geometry of the needle shroud to enable the distally directed lock surface to engage with the proximally directed locking geometry.

In some embodiments the resiliently movable lock element is structured as a lock sleeve.

Some embodiments of the autoinjector forms a device wherein the energy source comprises a helical compression spring arranged in a pre-tensed state exerting a distally directed force on the plunger.

In further embodiments a latch is associated with the memory element, the latch engaging when the memory element assumes the fired position to arrest the memory element in the fired position. In particular embodiments, the latch engages when the memory element assumes the fired position to permanently arrest the memory element in the fired position. In some embodiments the needle shroud is movable from the proximal collapsed position towards the distal extended locked position while the memory element is permanently arrested in the fired position.

The latch may in some embodiments be provided by cooperating latch geometries of the memory element and the housing to prevent the memory element from being moved distally away from the fired position. In other embodiments the latch is configured to rely on a frictional coupling, such as a frictional engagement, between the memory element and the housing to prevent the memory element from being moved distally away from the fired position. In some embodiments the latch is configured to provide a permanent axial locking of the memory element relative to the housing when the memory element assumes the fired position.

In some embodiments of the autoinjector, the device irreplaceably accommodates a container within the housing so that the container cannot be removed from the device without the use of tools. In such embodiments, the autoinjector forms a disposable device.

In some embodiments the container is provided as a syringe having a barrel and with an injection needle fixedly attached to the barrel.

In embodiments incorporating a cartridge and a separate needle unit, the cartridge and the needle unit may be initially held in a configuration where the cartridge and the needle unit are separated by a distance. The energy source may be capable, upon release of the plunger retaining arrangement, to cause the cartridge and the rear needle to enter into the state where the cartridge septum is pierced by the rear needle and subsequently to cause the plunger to move to dispense the drug through the needle.

In some embodiments the needle or needle unit substantially follows movement of the housing as the housing moves relative to the needle shroud. In particular embodiments, the needle/needle unit is attached to the housing in a way preventing relative axial movement between the housing and the needle/needle unit.

In a second aspect the present invention relates to an autoinjection device for expelling a dose of drug from a held drug container, the injection device comprising:
a housing having a proximal end (P) and a distal end (D),
a drug container comprising a container barrel and a piston that is sealingly and slideably arranged inside the container barrel,
an injection needle connected to or connectable to a distal end of the drug container,
a plunger adapted for cooperation with the piston to drive the piston distally along a central axis, the plunger comprising a retaining geometry,
an energy source coupled to the plunger and providing a force on the plunger in a distal direction,
a plunger retaining arrangement comprising a retaining element that engages with the retaining geometry to retain the plunger in a pre-firing position, the retaining element being movable in a radial direction to release said engagement,
a user operable trigger element cooperating with the retaining element and shiftable from a pre-firing position wherein the trigger element cooperates with the retaining element to maintain retaining engagement with the retaining geometry of the plunger, through an intermediate firing position wherein release of the retaining engagement is initialised, and further into a fired position,
   wherein the trigger element comprises an engagement surface configured for sliding engagement with an activation surface of the retaining element, wherein at least one of the engagement surface and the activation surface includes a surface being inclined relative to said radial direction, and
   wherein, upon the trigger element being shifted from the pre-firing position to the firing position, the energy source acts on the plunger to force the retaining element radially to release the retaining engagement, the radial movement of the retaining element in turn forcing the trigger element to move into the fired position by sliding engagement between the activation surface of the retaining element and the engagement surface of the trigger element.

In further embodiments according to the second aspect, any feature, or combination of features, mentioned above in connection with the first aspect may be provided in combination with the features in accordance with the second aspect.

As used herein, the term “drug” is meant to encompass any drug-containing flowable medicine or combinations of separately held plurality of drug-containing flowable medicines capable of being passed through a delivery means such as a cannula or hollow needle in a controlled manner, such as a liquid, solution, gel or fine suspension.

### DETAILED DESCRIPTION OF THE INVENTION

The invention will now be described in further detail with reference to the drawings in which:

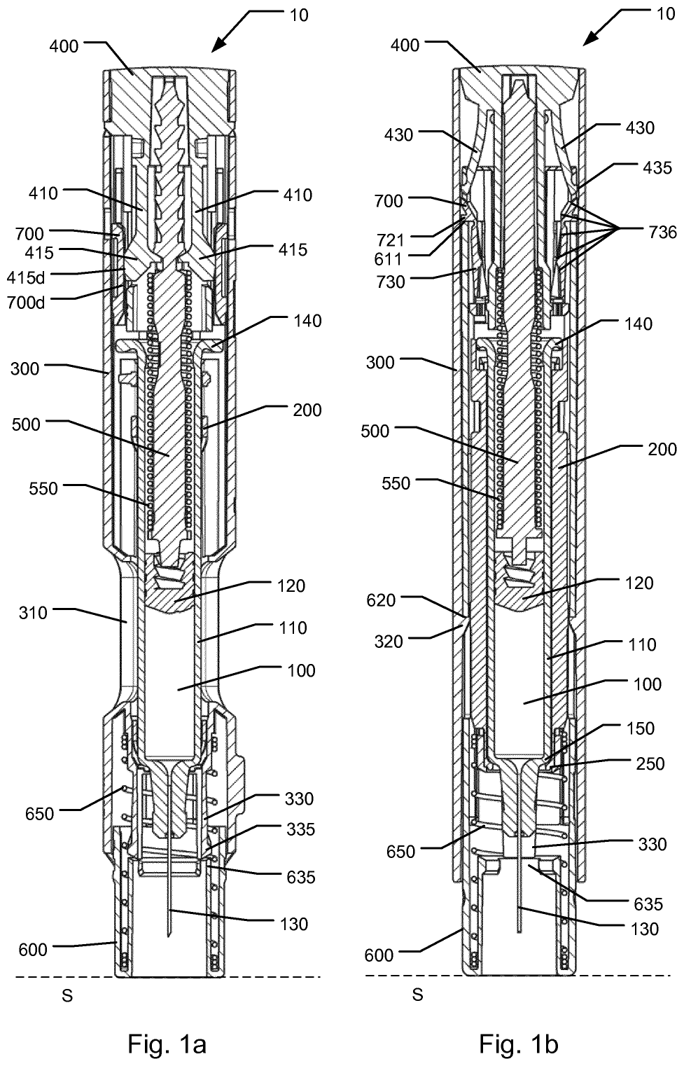

FIGS. 1a and 1b show sectional front and side views of an exemplary embodiment of an autoinjection device 10 according to the invention, the device being in a state where a needle shroud is fully extended and protects the needle of a held syringe,

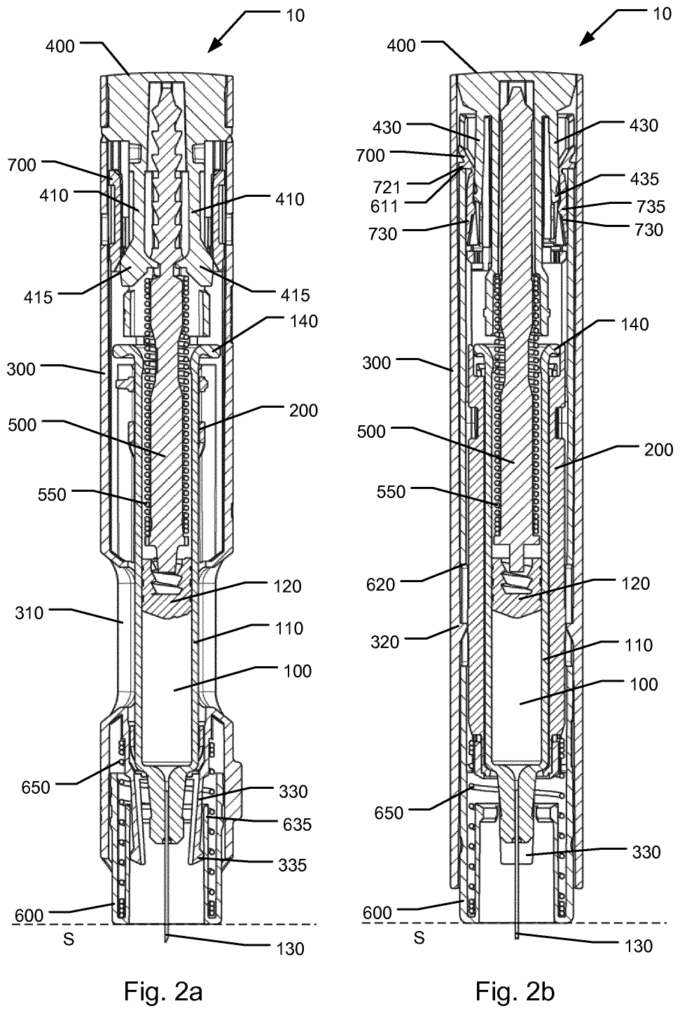

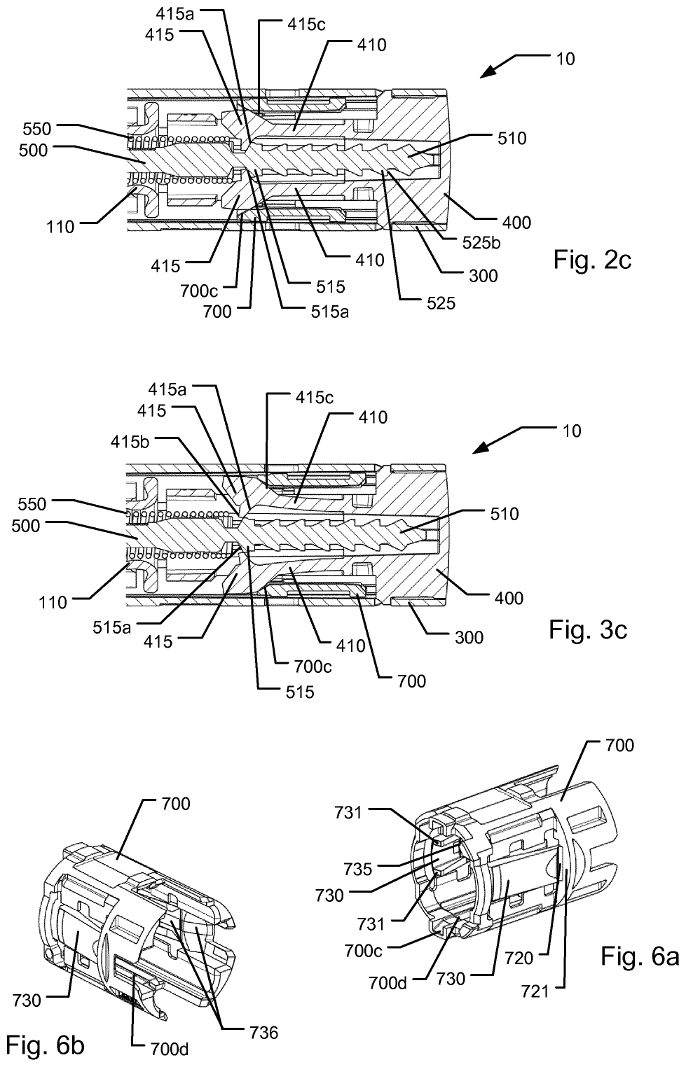

FIGS. 2a and 2b show sectional front and side views of the device 10 illustrating a state where the device has been pressed onto an injection site S and where an injection needle of a syringe initially protrudes from the needle shroud,

FIG. 2c is a detailed magnified view of FIG. 2a, showing the proximal portion of the device 10,

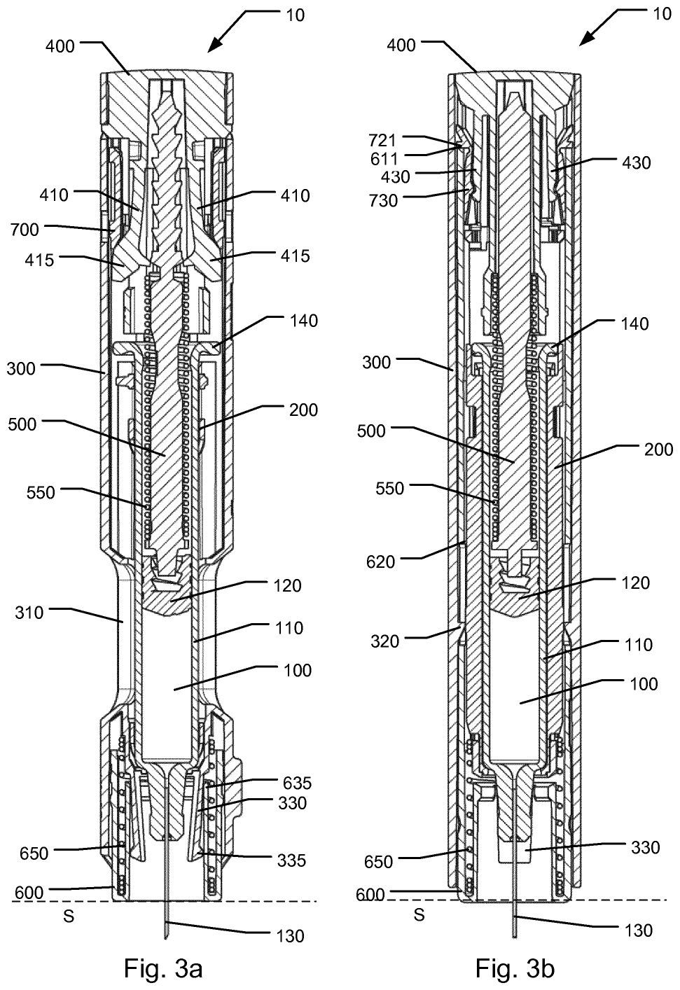

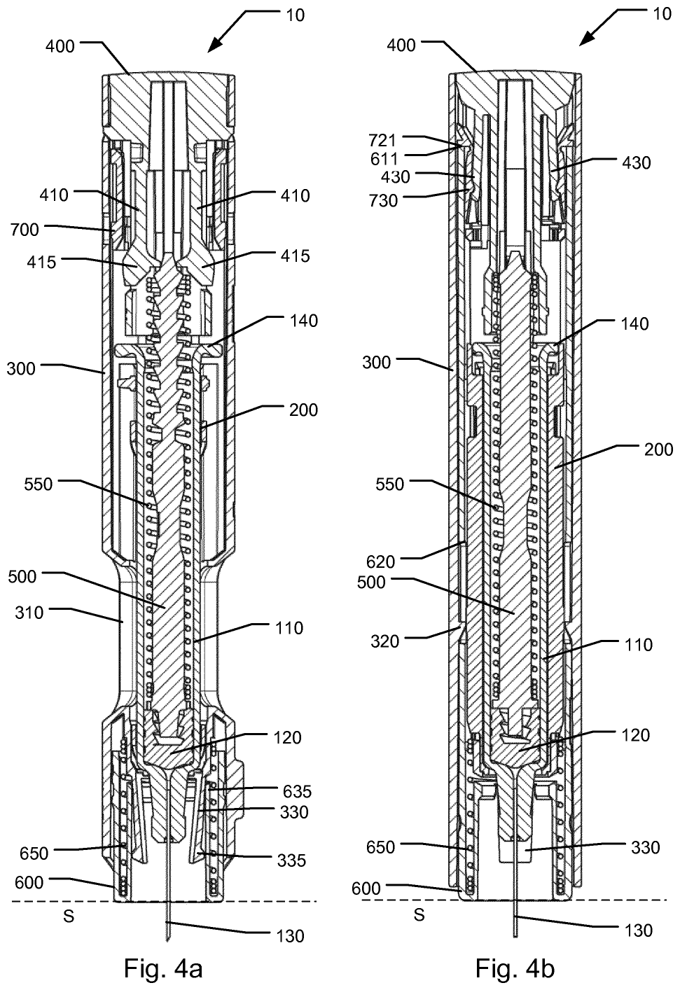

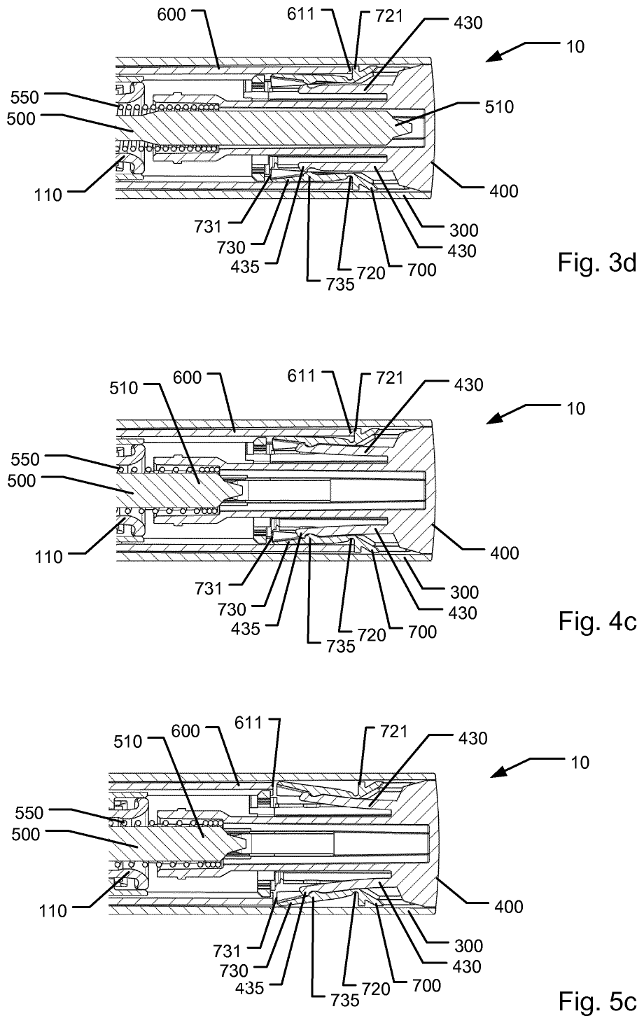

FIGS. 3a and 3b show sectional front and side views of the device 10 illustrating a state slightly before the needle protrudes fully from the needle shroud and wherein release of a plunger is about to be initiated,

FIG. 3c is a magnified view of FIG. 3a, showing the proximal portion of the device 10,

FIG. 3d is a detailed magnified view of the proximal portion of the device 10 in a view generally corresponding to the view shown in FIG. 3b but in a state just prior to the state shown in FIGS. 3a and 3b,

FIGS. 4a and 4b show sectional front and side views of the device 10 illustrating a state after the plunger has been released and where the drug of a held syringe has been expelled,

FIG. 4c is a detailed magnified view of FIG. 4b, showing the proximal portion of the device 10,

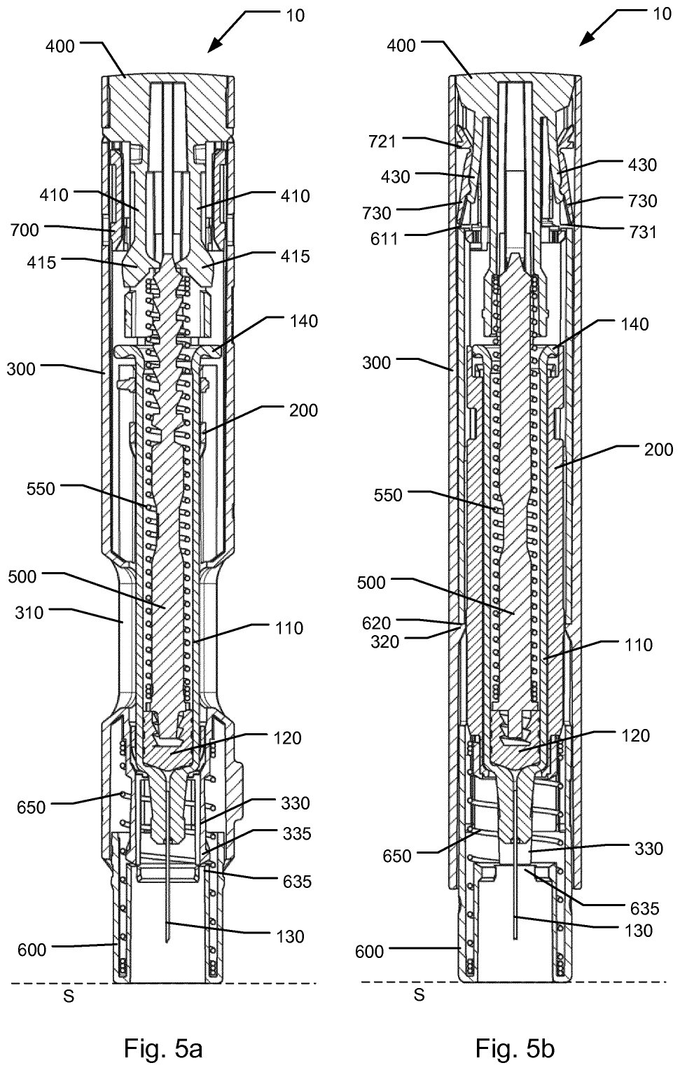

FIGS. 5a and 5b show sectional front and side views of the device 10 illustrating a state where the device has been lifted relative to the injection site S and wherein the needle shroud assumes a locked extended state,

FIG. 5c is a detailed magnified view of FIG. 5b, showing the proximal portion of the device 10, and

FIGS. 6a and 6b show perspective proximal and distal views of trigger element 700 of the injection device 10.

### DESCRIPTION

In the context of the present disclosure it may be convenient to define that the term “distal end” in the appended figures is meant to refer to the end of the injection device which carries the injection needle whereas the term “proximal end” is meant to refer to the opposite end of the injection device pointing away from the injection needle. The shown figures are schematical representations for which reason the configuration of the different structures as well as the relative dimensions are intended to serve illustrative purposes only.

The following is a description of an exemplary embodiment of a medical injection device 10 for administering a pre-determined amount of a liquid medicament. The device 10 is a disposable autoinjector configured for expelling a dose of a drug in a single administration whereafter the device 10 is ready for disposal. FIGS. 1a through 5c show various states of the injection device 10 during operation thereof with different views offering a detailed assessment of the operating principle.

Referring to FIGS. 1a and 1b, injection device 10 includes an elongated housing 300 that extends along a central longitudinal axis, housing being configured for being gripped by the palm of the user. The housing 300 forms a tubular shell which is closed off at the proximal end by a cap which in the following will be referred to as a power base 400. During assembly the power base 400 snaps into the housing 300 by means of snap protrusions which are received in recesses or openings to provide a non-releasable mounting of power base 400 within the proximal end of the housing 300.

At the distal end of the housing 300 a protective cap (not shown) will normally be arranged to cover a needle arrangement located at the distal end of the housing.

In the shown embodiment, the housing 300 accommodates a standard prefilled syringe (PFS) as widely used in industry. The syringe 100 comprises a tubular barrel 110 having a neck portion 115 located distally wherein the neck portion 115 has a reduced diameter compared to the diameter of the barrel 100. An injection needle 130 is mounted to the neck portion 115 and a removable cap (not shown) provided in the form of a rigid needle shield (RNS) will prior to use be attached to the neck 115 so that the needle shield sealingly and sterilely seals off the needle 130 Internally in the barrel 110 a slideably arranged piston 120 is arranged. A drug may be accommodated within the barrel between the piston 120 and the needle 130. Although the shown syringe only incorporates a single piston 120, other configurations may incorporate multiple pistons for accommodation and expelling of one or more drugs, including drugs to be reconstituted before administration. In other not shown embodiments, instead of a PFS type syringe, the housing may alternatively include other types of medicament containers, such as a cartridge configured to receive a separate injection needle.

Injection device 10 will typically be available in a form which further includes a removable protective cap (not shown) that attaches to a distal end of the device 10 to protect a needle end of the device 10. As commonly known for auto-injectors that incorporate a PFS syringe having an RNS shield attached, the protective cap may couple to the RNS so that the RNS is removed together with the protective cap. This situation is depicted in the state shown in FIGS. 1a and 1 b.

In the shown embodiment, a syringe holder 200 is arranged to hold syringe 100 inside housing 300 in a manner so that syringe 100 is fixedly withheld within the housing 300 by means of the syringe holder 200. Syringe holder 200 includes a body extending along a central longitudinal axis and being adapted to receive the barrel 110 of syringe 100. The body of the syringe holder 200 includes two longitudinal body sections disposed around the central longitudinal axis, where each of the body sections has a distal end with a radial inwards flange section 250 adapted for being received in a circumferential gap between the shoulder section 150 of barrel and the not shown RNS covering the needle. In this way the syringe holder 200 retains the syringe 100 to prevent the syringe from moving distally relative to the syringe holder 200. The two longitudinal body sections of syringe holder 200 are connected to each other by means of flexible portions allowing the two body sections to be radially moved away from each other to allow insertion of the syringe with the RNS attached into syringe holder 200. During manufacture, the assembly formed by the syringe holder and the syringe with the RNS attached is insertable into housing 300 through a proximal opening in the housing shell.

The lower distal half of the housing 300 includes two opposing window openings 310 allowing visual inspection of the drug contained within the syringe of the device 10. In addition, window openings 310 allow a user of the device to determine if the device 10 has been used for an injection by inspecting the presence or the location of a piston of syringe 100. During an injection, window openings 310 also allow for a rod-shaped plunger 500 of the device to become increasingly visible by the plunger gradually blocking more and more of the space between window openings 310.

FIGS. 1a and 1b show front and side sectional views of the device 10 after the protective cap has been removed but in a condition prior to the administration operation. Shown protruding from the distal end of housing 300 is a needle shroud 600 which is received partly within and arranged coaxially and axially slidable relative to housing 300. a needle shroud spring 650 is arranged biasing the needle shroud 600 in the distal direction. Needle shroud 600 is movable, when a proximally directed force is applied to the needle shroud 600, from a first distal extended position (shown in FIGS. 1a and 1b) and into a proximal collapsed position (shown in FIGS. 4a and 4b). Upon release of the proximally directed force, the needle shroud spring 650 pushes needle shroud 600 from the proximal collapsed position into a distal extended locked position (shown in FIGS. 5a and 5b).

The injection device 10 is configured for being triggered to expel a dose when the needle shroud 600 is moved from the distal extended position towards the proximal collapsed position. As the syringe 100 is substantially fixedly mounted within housing 300 of the device 10, the injection needle 130 follows axial movement of the housing when the housing is moved relative to the needle shroud 600.

The protective cap, when attached to injection device 10, prevents the needle shroud 600 from being manipulated and thereby prevents premature unintentional triggering of the injection device 10. In the shown embodiment, this function may be provided by a mechanism incorporating radially flexible arms 330 formed in the housing, the flexible arms having heads 335 formed at an internal location in the housing 300 arranged to cooperate with an internal skirt 635 provided inside the needle shroud 600. The heads 335 and skirt 635 are located at the same radial position. Thus, for the needle shroud 600 to become pushed proximally relative to housing 300, the flexible arms 330 with heads 335 are required to become deflected radially inwards by cooperating with skirt 635 before the skirt and thus the entire needle shroud is movable away from the distal extended position. As long as the RNS and/or the protective cap is still attached to the syringe the flexible arms 330 with heads 335 are initially blocked against moving radially inwards by the presence of the RNS.

The skirt 635 is thus not able to axially pass the heads 335. Only after removal of the protective cap with the RNS and forcing the needle shroud 600 towards the proximal collapsed position the heads will cooperate with skirt 635 to move the heads radially inwards and allow the skirt 635 to pass the heads of the flexible arms (cf. FIG. 2a).

Piston 120 is driveable towards the needle outlet in order to dispense medicament from the syringe 100. The dispensing is carried out by an expelling assembly incorporating the plunger 500 and a pre-stressed drive spring 550.

In the shown embodiment, the needle shroud 600 forms a distal portion and a proximal portion. The distal portion is provided as a generally hollow tubular member having a distal end rim arranged to form an abutment surface, the tubular member initially covering the injection needle 130. The proximal portion of the needle shroud 600 forms two opposed axial extending legs extending from the distal portion and in the proximal direction for a substantive part of the length of the housing. Each of the two opposed axial extending legs ends in a proximally facing abutment surface 611. The needle shroud 600 with its two legs is shaped to be accommodated within the housing 300 with the radial outer surface of the needle shroud being in intimate but slideable contact with a radially inwards facing cylindrical surface of the housing shell.

The needle shroud 600 cooperates with a trigger element 700 which is located at the proximal end of the needle shroud 600. Trigger element 700 serves as a memory element which assumes a first distal position prior to use of the device 10, and which assumes a second proximal pre-defined parked position after the device 10 has been fully triggered, and wherein the memory element stays in the parked position subsequent to triggering. In the shown embodiment the trigger element both serves as a trigger sleeve, and also serves as a lock sleeve for the needle shroud. For accommodating both functions, the trigger element 700 is movable axially in the proximal direction relative to the housing 300 from a pre-firing position (FIGS. 1a and 1 b) to a firing position (FIGS. 2a and 2b), and further to a fired position (FIGS. 4a and 4b). Trigger element 700 is formed as a generally tubular hollow member. Initially, before triggering, and until the needle shroud 600 is pushed distally after expelling, a proximal portion of the legs of the needle shroud 600 is arranged in axially overlapping relationship with the trigger element 700. In this relative position the trigger element 700 is accommodated radially between the two opposed axial extending legs of the needle shroud 600 so that an inner surface of each leg is in slideable contact with a radial outwards facing surface of the trigger element 700. Referring to the state shown in FIG. 1b, while also referring to FIG. 6a, each proximally facing abutment surface 611 of the legs of the needle shroud 600 abuts a distal facing abutment surface 721 of the trigger element. Thus, when the needle shroud 600 is moved from its distal extended position and into its proximal collapsed position, the trigger element 700 is slaved for moving from the pre-firing position to the firing position. As will be described further below, the trigger element 700 is moved further into its fired position by means of a proximal directed force emanating from the drive spring 550, optionally accompanied by a proximal directed force exerted by the needle shroud 600.

In the shown embodiment, both the needle shroud 600 and the trigger element 700 are mounted in a way that prevents rotational movement but allows axial movement relative to the housing 300. The needle shroud 600 is urged in the distal direction by means of the needle shroud spring 650 so that when no externally applied force is exerted on the needle shroud, the needle shroud assumes its distal extended position which is shown in FIGS. 1a and 1b. In this position a stop geometry 620 on needle shroud 600 engages a stop 320 in the housing preventing the needle shroud 600 from moving further in the distal direction. When an externally applied force is exerted on the needle shroud 600 for moving the needle shroud in the proximal direction relative to the housing, such as when device 10 is pressed with the needle shroud against an injection site, the externally applied force acts counter to the force provided by the needle shroud spring 650 resulting in the needle shroud 600 and the trigger element 700 being forced to move in the proximal direction relative to the housing. When the needle shroud 600 assumes the proximal collapsed position a proximal facing surface of the trigger element 700 prevents the trigger element and thus the needle shroud 600 from moving further proximally relative to the housing.

As the device 10 is removed from the injection site, the needle shroud 600 will move distally due to the force from the needle shroud spring 650. After an injection has been performed, as the needle shroud 600 reaches its distal extended position again, as shown in FIGS. 5a and 5b, it will be locked in this position to render the needle shroud inoperable (to be further explained below). While referring to “its distal extended position” it is to be noted that the shown device 10 is so designed that the said distal extended position where the needle shroud is made inoperable corresponds to the initial distal extended position the needle shroud assumes prior to triggering. However, in other embodiments, the final distal extended position where the needle shroud is made inoperable may be located slightly different than the initial distal extended position prior to triggering, e.g. positioned at a position slightly proximally or slightly distally relative to the distal extended position shown in FIGS. 1a and 1b.

The needle 130 of syringe 100 is arranged at the distal end of the housing 300, such that the needle shroud 600 completely covers the needle when the needle shroud is in its distal extended position. When the needle shroud 600 is in its proximal collapsed position, the needle 130 protrudes through a central opening in the needle shroud 600.

The expelling assembly of injection device 10 is based on a plunger that is driven in the distal direction along the central longitudinal axis of the device for advancing the piston 120 to thereby expel the dose of drug accommodated within the syringe 100. The plunger 500 in the shown embodiment forms a solid rod having a circular flange arranged at the distal end of the plunger. In device 10 with the rod-shaped plunger 500 arranged along the central axis, a stored energy source in the form of a pre-stressed helical compression drive spring 550 is arranged to encircle the plunger rod 500 along a portion of its length. Drive spring 550 is energized by straining the compression spring during manufacture of the device. The distal end of drive spring 550 is supported onto plunger 500 by a circular flange arranged at the distal end of the plunger. The proximal end of drive spring 550 is supported by a spring seat (non-referenced) formed at a distal end of power base 400 and thus grounds the proximal end of drive spring relative to the housing 300.

As mentioned, in the shown embodiment, the drive spring 550 urges the plunger 500 in the distal direction. In the non-triggered state of the injection device 10, a plunger retaining arrangement associated with the housing engages with a retaining geometry of the plunger to retain the plunger 500 in a pre-firing position. In the shown embodiment, and referring to 1a, FIGS. 2c and 3c, the retaining arrangement comprises, on the plunger 500, a pair of stepped blocking geometries 515 proximally adjoining a recessed portion of the plunger 500. The plunger retaining arrangement further comprises two retaining arms 430 extending axially in the distal direction from the power base 400. Each of the two retaining arms 430 forms a radially resilient arm that ends in an enlarged blocking head 415 having its radially inwards facing portion situated in the recessed portion of the plunger rod 500. Generally referring to FIG. 2c, with the plunger retaining arrangement assuming the state shown in FIG. 1a, an inclined proximal surface 415a of enlarged blocking head 415 engages a correspondingly inclined distal surface 515a. Hence, the force exerted by drive spring 550 acts to push the enlarged blocking heads 415 radially outwards. In the initial non-triggered state of the device 10, as shown in FIG. 1a, a radially outward facing surface 415d provided on each of the enlarged blocking heads 415 engages a radially inwards surface 700d of the trigger element 700, the presence of the trigger element, when located in the pre-firing position, thus effectively prevents triggering of device 10.

Referring to FIG. 2c, each of the enlarged blocking heads 415 includes, at a radially outwards portion thereof, an inclined proximally facing surface 415c configured for sliding engagement with a corresponding inclined distal facing surface 700c provided at the distal end of the trigger element (see FIGS. 2c and 6a). During triggering, the trigger element 700 will initially be pushed proximally by the needle shroud 600, due to engagement between elements 611 and 721, and once the trigger element assumes the firing position shown in FIG. 2c, the proximally facing surfaces 415c of enlarged blocking heads 415 axial aligns and engages the inclined distal facing surfaces 700c of trigger element 700. Due to the inclination, the radially outward force provided by the drive spring onto the enlarged blocking heads 415 will act to urge the trigger element 700 further proximally meaning that the force provided by needle shroud 600 to initiate the trigger movement of trigger element 700 will be amplified by the force provided by the drive spring 550. This causes the trigger element to be effectively pushed proximally until the trigger element 700 assumes the fired position shown in FIG. 3c. During movement of trigger element 700 from the firing position shown in FIG. 2c and into the fired position shown in FIG. 3c, the axial movement of trigger element is likely to be accompanied by proximal movement of the needle shroud 700 due to the momentum of movement of the needle shroud during the triggering movement of needle shroud.

As shown in FIG. 3c, the state refers to a situation where, for each resilient arm 430, the inclined proximal surface 415a of enlarged blocking head 415 slips free from engagement relative to the inclined distal surface 515a on the plunger 500, and the plunger 500 is thus released to be driven forward by the drive spring 550.

Alternatively to using a pre-stressed spring which is compressed during manufacture of the device, other embodiments of autoinjectors in accordance with the present disclosure may include a mechanism for compressing the spring as an initial procedure when putting the device into use. Also, the energy source may in other embodiments be provided as a torsion spring which is pre-stressed to exert a torsion force for driving forward a rotational drive of the expelling assembly. Alternatively, the energy source may be in the form of a compressed medium such as a gas. Still alternatively, the energy source may include a gas generator such as an electro-chemical cell.

Referring again to FIG. 2c, the plunger 500 furthermore provides, at its proximal portion, a series of teeth 525 each having a gradually rising slope and an abrupt decline in the direction of relative movement. A radially inwards facing surface of the enlarged blocking head 415 of each resilient arm 430 is configured to sequentially cooperate by the inherent elastic properties of the resilient arm with each tooth 525 to generate an audible click as the enlarged blocking head passes each tooth. In the shown embodiment, each of the enlarged blocking heads 415 cooperate with six consecutive teeth. The enlarged blocking heads and the teeth thus generate progress clicks in the course of the dispensing procedure to signal expelling of liquid drug, and with the omission of a click to signify end of dosing. In the shown embodiment, two opposed retaining arms are provided that cooperate with a corresponding number of protrusions or recesses formed on the plunger. In other embodiments a single arm may be provided necessitating a support surface of some kind arranged radially oppositely to the single arm. In still other embodiments, three or more arms may be provided, preferably being disposed symmetrically around the axis.

In the following, the components that relate to the needle shroud lock function will be further described. Referring back to FIG. 1b, 5c and FIG. 6a/6b, the trigger element 700 includes two resiliently movable lock elements formed as a pair of deflectable lock arms 730 forming part of the needle shroud lock mechanism. When the needle shroud lock function is established, the deflectable lock arms 730 render the needle shroud 600 permanently arrested, i.e. when the needle shroud, subsequent to finalisation of an injection, is returned to the distal extended position. As shown in FIG. 6a, each of the deflectable arms 730 connects by means of a film hinge 720 to the remaining of the trigger element 700. Each of the deflectable arms 730 comprises a rigid beam section extending from the film hinge 720 to a free distal end comprising distally directed lock surfaces 731. The deflectable arms 730 are due to the film hinge 720 able to be moved radially outwards from a non-locking position where the locking arms 730 lie flush with the neighbouring surfaces of trigger element 700 and into a locking position where the lock surfaces 731 extend radially outwards from said neighbouring surfaces.

In the shown embodiment, the needle shroud lock function further incorporates the power base 400. Power base 400 additionally includes two independent flexible arms 430 each extending in the distal direction from the power base. Each flexible arm is biased radially outwards so that a latch head 435 provided at the free distal end of the flexible arm assumes the position shown in FIG. 1b wherein the latch head 435 radially abuts the inner tubular surface of housing 300. Each latch head 435 comprises a radially outwards facing surface that is configured to cooperate by resiliently sliding against corresponding profiled axial tracks 736 of trigger element 700.

As shown in FIGS. 1b and 6a/6b, the deflectable arms 730 assume an unbiased non-locking radial position when the deflectable arms are not engaged by latch heads 435 of the two flexible arms 430. Each of the latch heads 435 of the two flexible arms 430 are configured to provide a radially outwards directed force on the corresponding deflectable arm 730 when the locking sleeve 700 is situated in the fired position, i.e. as shown in FIG. 4c. In this position each of the latch heads 435 grips behind a one-way latch protrusion 735 residing in the profiled axial track 736. Thus, when the trigger element 700 assumes the fired position the trigger element is arrested in the fired position and is prevented from moving distally again by latching engagement between latch heads 435 and one-way latch protrusions 735. For comparison the state shown in FIG. 3d provides a view of the device 10 just prior to the trigger element in the firing position where the latch heads 435 are positioned proximally relative to the one-way latch protrusions 735 and thus latch heads 435 are not yet latched. In both the firing position and in the fired position the flexible arms 430 of the power base 400 provides a radially outwards biased force onto the corresponding deflectable arm 730 of the trigger element. As the legs of the needle shroud 600 are positioned between the deflectable arms 730 and the housing 300 the deflectable arms 730 are still prevented from moving radially outwards into their locking position.

FIGS. 4a and 4b provide views of the device 10 in a state where the trigger element 700 is in the fired position, and the plunger 500 has already been caused to expel the dose of the syringe by plunging forward the piston 120 of syringe 100. The series of progression clicks have been generated during expelling and the piston has bottomed out in syringe barrel 100. The latch heads 435 of power base 400 grips behind the one-way latch protrusions 735 and. Thus, trigger element 700 is arrested in the fired position.

When the device 10 is removed from the injection site S the needle shroud spring 650 forces the needle shroud 600 from the proximal collapsed position into the distal extended locked position. During this movement, the proximally facing abutment surfaces 611 of the legs of the needle shroud initially moves out of engagement with the distal abutment surfaces 721 of the trigger element 700. Continued distal movement makes the legs of the needle shroud slide along the trigger element 700 until the proximally facing abutment surfaces 611 axially align with the distally directed lock surfaces 731 of the deflectable arms 730. Due to the radially outwards biased force from the flexible arms 430 onto the cooperating deflectable arms 730, the deflectable arms 730 are forced to move radially outwards into their locking position. As a consequence, the distally directed lock surfaces 731 of the deflectable arms 730 enter into blocking position relative to the proximally facing abutment surfaces 611 of the legs of the needle shroud 600 and the needle shroud 600 is prevented from moving towards the proximally collapsed position after the device 10 has been triggered.

Returning now briefly to details which relate to the triggering procedure of injection device 10 wherein the needle shroud 600 and the trigger element is moved in the proximal direction relative to the housing 300. Due to the profiled nature of axial tracks 736 the latch heads 435 initially climb a steep portion of the profiled axial tracks 736 (climb meaning move in the radial direction). This creates an initially high force which must be overcome by the user when pushing device 10 against an injection site S to make the needle shroud 600 move towards the proximal collapsed position.

When the trigger element 700 is moved from the distal extended position towards the proximal collapsed position the two flexible arms 430 and the corresponding profiled axial tracks 736 of the trigger element 700 provide resistance to movement of the trigger element 700 and thus also resistance to movement of the needle shroud 600. Upon applying the autoinjector 10 at an injection site, a high axial reaction force is initially created when the flexible arms 430 engage the proximal end portion of the profiled axial tracks 736. Thus, a high force exerted on the needle shroud 600 is required in order for the flexible arms 430 to climb the profiled axial tracks 736. As soon as the flexible arms 430 have climbed the profiled axial tracks 736, resulting in the flexible arms 430 have been deformed radially inwards, the flexible arms 430 travel and slide along an almost constant height track profile as the needle shroud 600 is pushed further proximally relative to housing 300. This action requires considerable less force to be applied on the needle shroud 600 than the initial high force. Hence the needle shroud displacement will occur in two stages, i.e. a first high force stage and a second low force stage.

It will be appreciated, that the force needed for proximally displacing the needle shroud will be largely independent from the force provided by the drive spring, but will rather be decided by the force of the needle shroud spring 650 and the force profile for the interaction between the flexible arms 430 and the profiled axial tracks 736. A further minor force which must be overcome when pushing in the needle shroud 600 emanates from the flexible arms 330 of the housing cooperating with the inner tubular proximal rim 635 of the needle shroud 600, cf. the discussion mentioned above with respect to the removal of the protective cap/RNS.

As will be discussed further below, the above-mentioned firing position of trigger element 700, and the corresponding position of needle shroud 600, will be situated at the final part of the proximal needle shroud movement where the flexible arms 430 travel along the almost constant height profile of axial tracks 736. The high initial needle shroud force over a short distance assures that the needle shroud is fully displaced and the autoinjector is effectively triggered due to the inertia of the human motion.

Some preferred embodiments have been shown in the foregoing, but it should be stressed that the invention is not limited to these, but may be embodied in other ways within the subject matter defined in the following claims.

1. An autoinjection device for expelling a dose of drug from a held drug container, the injection device comprising:
a housing having a proximal end (P) and a distal end (D),
a drug container comprising a container barrel and a piston that is sealingly and slideably arranged inside the container barrel,
an injection needle connected to or connectable to a distal end of the drug container,
a plunger adapted for cooperation with the piston to drive the piston distally along a central axis, the plunger comprising a retaining geometry,
an energy source coupled to the plunger and providing a force on the plunger in a distal direction,
a plunger retaining arrangement comprising a retaining element that engages with the retaining geometry to retain the plunger in a pre-firing position, the retaining element being movable in a radial direction to release said engagement,
a user operable trigger element cooperating with the retaining element and shiftable from a pre-firing condition wherein the trigger element cooperates with the retaining element to maintain retaining engagement with the retaining geometry of the plunger, and into a firing condition wherein release of the retaining engagement is initialised,
wherein the autoinjection device defines a memory element being movable from a pre-firing position to a fired position, the memory element comprising an engagement surface configured for sliding engagement with an activation surface of the retaining element, wherein at least one of the engagement surface and the activation surface includes a surface being inclined relative to said radial direction, and
wherein, upon the trigger element being shifted from the pre-firing condition to the firing condition, the energy source acts on the plunger to force the retaining element radially to release the retaining engagement, the radial movement of the retaining element in turn forcing the memory element to move into the fired position by sliding engagement between the activation surface of the retaining element and the engagement surface of the memory element, said movement of the memory element being induced by said surface being inclined relative to said radial direction.

2. The autoinjection device as defined in claim 1, wherein the memory element is axially movable, and wherein said at least one surface is inclined relative to said radial direction and so oriented as to induce axial movement of the memory element from the firing position to the fired position upon radial movement of the retaining element.

3. The autoinjection device as defined in claim 1, wherein the memory element is rotationally movable, and wherein said at least one surface is inclined relative to said radial direction and so oriented as to induce rotation of the memory element from the firing position to the fired position upon radial movement of the retaining element.

4. The autoinjection device as defined in claim 1, wherein the retaining element includes a retaining surface that engages a cooperating surface of the retaining geometry to retain the plunger in the pre-firing position, and wherein one or both of the retaining surface and the cooperating surface include(s) a surface being inclined relative to said radial direction so that distal movement of the plunger, upon initial release of the retaining engagement, induces radial movement of the retaining element to disengage the retaining surface from the cooperating surface of the retaining geometry.

5. The autoinjection device as defined in claim 1, wherein the trigger element assumes a pre-firing position when the trigger element assumes the pre-firing condition, and assumes a firing position when the trigger element assumes the firing condition, and wherein the trigger element cooperates with the retaining element to initiate release of the retaining engagement when the trigger element assumes the firing position.

6. The autoinjection device as defined in claim 5, wherein the trigger element defines said memory element, and wherein the trigger element is movable from the pre-firing position to the firing position, and further to the fired position.

7. The autoinjection device as defined in claim 6, wherein the autoinjection device comprises a needle shroud being axially movable relative to the housing, and a needle shroud spring which is arranged biasing the needle shroud in the distal direction, wherein the needle shroud is movable from a first distal extended position into a proximal collapsed position when a proximally directed force is applied to the needle shroud, and from the proximal collapsed position into a distal extended locked position, and wherein the trigger element couples to the needle shroud so that the trigger element moves from the pre-firing position to the fired position in response to the needle shroud being moved from the first distal extended position into the proximal collapsed position.

8. The autoinjection device as defined in claim 7, wherein when the needle shroud moves from the first distal extended position towards the proximal collapsed position the needle shroud causes the trigger element to move from the pre-firing position into the fired position, the needle shroud slaving the trigger element into the firing position, and optionally into the fired position.

9. The autoinjection device as defined in claim 8, wherein a latch is associated with the trigger element, the latch engaging when the trigger element assumes the fired position to arrest the trigger element in the fired position.

10. The autoinjection device as defined in claim 9, wherein, with the trigger element arrested in the fired position, when the needle shroud moves from the proximal collapsed position into the distal extended locked position, the needle shroud moves relative to the arrested trigger element and wherein the needle shroud cooperates with the trigger element to lock the needle shroud as the needle shroud is moved distally into the distal extended locked position.

11. The autoinjection device as defined in claim 10, wherein at least one of the needle shroud and the trigger element comprises a lock element which is resiliently urged towards the other of the needle shroud and the trigger element to move along relative to a surface of said other of the needle shroud and the trigger element when the needle shroud moves relative to the arrested trigger element in the fired position until the lock element reaches a locking geometry formed in or on said other of the needle shroud and the trigger element upon the needle shroud being moved distally into the distal extended locked position so as to lock the needle shroud in the distal extended locked position.

12. The autoinjection device as defined in claim 11, wherein the distally directed lock surface is formed on a resiliently movable lock element being movable from a non-locking position into a locking position, and wherein a biasing structure urge the resiliently movable lock element towards moving to the locking position, and wherein the resiliently movable lock element is moved from the non-locking position into the locking position upon the needle shroud being moved from the proximal collapsed position into the distal extended locked position.

13. The autoinjection device as defined in claim 12, wherein the resiliently movable lock element is configured to slide along a sliding surface of the needle shroud as the needle shroud is moved from the proximal collapsed position into the distal extended locked position for the distally directed lock surface of the trigger element to axially align with the proximally directed locking geometry of the needle shroud to enable the distally directed lock surface to engage with the proximally directed locking geometry.

14. The autoinjection device as defined in claim 1, wherein the energy source comprises a helical compression spring arranged in a pre-tensed state exerting a distally directed force on the plunger.

15. The autoinjection device as defined in claim 1, wherein a latch is associated with the memory element, the latch engaging when the memory element assumes the fired position to arrest the memory element in the fired position.
